(19) United States
(12) Patent Application Publication
(10) Pub. No.: US 2004/0193284 A1
(43) Pub. Date: Sep. 30, 2004

(54) Myometrium as a source for cardiomyoplasty

(76) Inventor: Syde A. Taheri, Williamsville, NY (US)

Correspondence Address: Walter W. Duft, Suite 10, 10255 Main Street, Clarence, NY, 14031, US

(21) Appl. No.: 10/396,742

(22) Filed: Mar. 25, 2003

(51) Int. Cl. A61F 2/02; C12N 5/08

(52) U.S. Cl. 623/23.72; 623/904; 435/366

(57) ABSTRACT

A cardiomyoplasty method for treating cardiomyopathy using a myometrial compostion of myometrial tissue, and a device for implantation without injection into the myocardium. The myometrial composition may include cut, granulated, ground or cultured myometrial tissue. The myometrial composition may also be treated with one or more additives such as cDNA vascular endothelial growth factor (VEGF), a patient's blood, or an adhesive. A first implantation device includes a tubular double lumen body having a first and second pathway, a spray opening adapted to deliver the myometrial composition, and a pressure valve to release unsafe pressure within the pericardium. A second implantation device includes a tubular lumen outer body and a first and second tubular lumen inner bodies. The first inner body has an expandable portion to carry a myometrial tissue patch. The expandable portion may include longitudinal slots to carry the myometrial composition.

### BACKGROUND OF THE INVENTION

1. Field of the Invention

This invention relates to cardiomyopathy and the treatment thereof.

2. Description of the Prior Art

By way of background, cardiomyopathy, as caused for example by myocardial infarction, is a common disorder. Twenty thousand new cases are reported yearly in the United States and 25-50% of such cases will result in death after three years. The problem is that damaged adult heart muscle does not regenerate and myocardial functionality cannot be restored using the body's natural healing mechanisms. The myocardium tends to dilate and areas of the ventricular walls may become hypokinetic, or even akinetic, such that congestive heart failure often develops in affected individuals.

Previous medical techniques have not substantially reduced the morbidity or mortality of this condition. Past efforts in this area include removing autologous or allogeneic skeletal muscle cells or stem cells, and culturing them to generate the large number of implantation cells necessary for myocardial repair. The cultured cells are then implanted via injection into the myocardium, where they have an opportunity to regenerate new heart muscle. Applicant has also previously proposed a cell patch method whereby autologous skeletal muscle grafts are applied to damaged myocardial tissue without culturing. See U.S. Pat. No. 5,327,913. According to this method, skeletal muscle grafts are obtained and placed against a patient's outer myocardial wall. A section of the patient's greater omentum is applied over the grafts to supply blood to the transplanted tissue. In a subsequently developed improvement of the cell patch method, a percutaneous procedure is used to secure the muscle grafts to the myocardium. See U.S. Pat. No. 6,435,190. Applicant has also proposed a micro-granule treatment wherein autologous myocyte micro-granules are injected into a patient's inner or outer myocardial wall using an injection needle introduced via a transfemoral or surgical approach. According to a further proposal by Applicant, a cradle-assisted myocardial repair and treatment method uses a cradle designed for engagement with the myocardium and injection of a myocyte donor material therein. The cradle supports injection needles for injecting the desired myocardial repair or treatment material into the myocardium. It is to improvements in the treatment of cardiomyopathy that the present invention is directed. In particular, the present invention builds on Applicant's previous work by proposing a new source of myocardial repair or treatment material, with advantage being provided by functional properties of the material that differentiate it from previous repair or treatment compositions.

### SUMMARY OF THE INVENTION

The foregoing problems are solved and an advance in the art is obtained by a novel cardiomyoplasty method for treating cardiomyopathy using myometrial tissue, a myometrial composition for cardiomyoplasty, and a device for myometrial tissue implantation without injection into the myocardium. In accordance with exemplary embodiments of the invention, myometrial tissue is extracted from a donor area in a myometrium using an appropriate instrument or device. The extracted myometrial tissue is then processed for cardiomyoplasty implantation. An opening is formed in the pericardium to expose an implantation area on the patient's myocardium where damaged myocardial tissue is located. The processed myometrial tissue is then applied to the designated implantation area.

Processing of the myometrial tissue may include cutting, culturing, granulating or the like. The myometrial tissue may also be treated with one or more additives such as cDNA vascular endothelial growth factor (VEGF), a patient's blood, and/or a suitable adhesive to form the desired myometrial composition.

The myometrial tissue implantation device is used to implant the myometrial composition in the damaged area of the patient's myocardium. One exemplary embodiment of this device includes a tubular double lumen body having a first pathway and a second pathway. The body is constructed with a spray opening at a proximal end of the first pathway adapted to deliver the myometrial composition. The second pathway has a pressure valve to release any unwanted pressure within the pericardium that could occur during implantation. The body also includes an expandable stabilizer that secures the spray opening in position during implantation.

Another exemplary novel mymometrial tissue implantation device includes a tubular lumen outer body, a first tubular lumen inner body, and a second tubular lumen inner body. The first inner body has an expandable portion at a proximate end adapted to carry a myometrial tissue patch. The expandable portion may include longitudinal slots adapted to carry a myometrial composition of cells or granules. The second inner body has a plunger slidably disposed therein to deliver a securing member to the implanted tissue.

### BRIEF DESCRIPTION OF THE DRAWINGS

The foregoing and other features and advantages of the invention will be apparent from the following more particular description of preferred embodiments of the invention, as illustrated in the accompanying Drawings, in which:

FIG. 1 is a plan view of a human torso showing a uterine donor area with myometrium tissue;

FIG. 2 is a perspective view showing a cannula and guide wire being placed through a pericardial sac and proximate to a damaged myocardial area;

FIG. 3 is a perspective view including enlarged inset illustrations “A” and “B” of an exemplary percutaneous myometrial implantation device constructed in accordance with the invention showing the implantation system entering an opening in a dilated pericardial sac to treat a damaged myocardial area;

FIG. 4 is a perspective view of the implantation device of FIG. 3 positioned proximate to a damaged myocardial area and applying a myometrial composition to the myocardium;

FIG. 5 is a perspective view of an outer body of a second exemplary percutaneous myometrial implantation device proximate to a damaged myocardial implantation area;

FIG. 6 is a perspective view of a first inner body of the myometrial implantation device of FIG. 5;

FIG. 7 is a perspective view of the first inner body of FIG. 5 with myometrial cells carried thereon;

FIG. 8 is a perspective view including an enlarged inset illustration “C” of the outer body and first inner body of FIG. 5 with a myometrial tissue patch attached to the first inner body;

FIG. 9 is a perspective view of the outer body and first inner body of FIG. 5 and attached myometrial tissue patch proximate to the implantation area;

FIG. 10 is a perspective view of the outer body and first inner body of FIG. 5, the attached myometrial tissue patch, and myometrial cells being released proximate to the implantation area;

FIG. 11 is a perspective view of the outer body of FIG. 5 and the myometrial tissue patch being positioned onto the implantation area using guide wires;

FIG. 12 is a perspective view of the outer body of FIG. 5, the myometrial tissue patch, and a second inner body;

FIG. 13 is a perspective view of the outer body of FIG. 5 and the second inner body being used to detach the guide wires from the positioned myometrial tissue patch;

FIG. 14 is a perspective view including an enlarged inset illustration “D” of the outer body of FIG. 5, the second inner body, a plunger, and a securing tack;

FIG. 15 is a perspective view of the outer body of FIG. 5 and the myometrial tissue patch partially attached to the implantation area with the securing tack;

FIG. 16 is a perspective view of the myometrial tissue patch fully attached to the implantation area with securing tacks;

FIG. 17 is a perspective view of a fully attached mymometrial tissue patch treated with myometrial cells.

### DETAILED DESCRIPTION OF THE PREFERRED EMBODIMENT

Use of the myometrium as a source for cardiomyoplasty will now be described by way of exemplary embodiments shown by the drawing figures, in which like reference numerals indicate like elements in all of the several views.

Presently, cell transplantion is performed in most cases using skeletal muscle culture or cell patch. A problem with this type of cardiomyoplasty is that skeletal muscle lacks the Connexine 43 proteins to transmit the proper signals to synchronize contraction. Accordingly, a need exists for tissue or cells for cardiomyoplasty transplant that will initiate a coordinated contraction with the rest of the myocardial cardiomyocytes. The uterus is an organ that displays such contraction, as can be seen during childbirth. Myometrium, the uterine smooth muscle, is the only smooth muscle that contains Connexine 43 with coordinated strong contraction. An abundance of capillaries and synchronization of contraction to an infarcted myocardium makes the myometrium a unique muscle to consider for transplant.

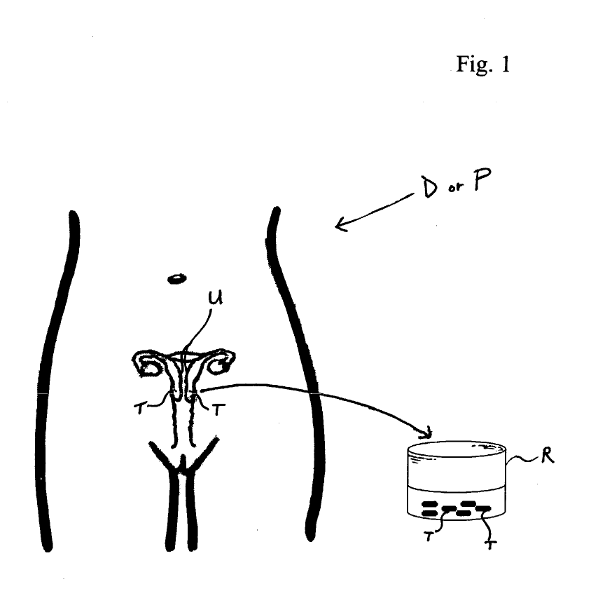

Turning to FIG. 1, a transvaginal biopsy of myometrial tissue T is performed on a donor D (such as a hysterectomy patient), or a patient P whose myocardium is to be repaired. Note, in the case of a donor D, the myometrial tissue T is used when the patient P is a Human Leukocyte Antigen (H.L.A.) match with the tissue such that it is allogenic. The myometrial tissue T is retrieved by excising it from a uterine wall U and placed in a sterile storage receptacle R containing a suitable medium such as a saline solution. Next, the myometrial tissue T is processed and prepared for implantation as a myometrial composition in a manner now to be described. The myometrial tissue T can remain intact or it can be homogenized and cultured as individual cells or a population of cells, or it can be cut or ground into granules or other fragments. The size of the myometrial tissue T that comprises the myometrial composition may thus range from single cell size to multi-cell clumps on the order of about 3-5 mm along the largest dimension. The myometrial composition may also include an additive such as a patient's blood, cDNA vascular endothelial growth factor (VEGF) originating from the patient P, and/or a suitable adhesive, such as serum albumin/glutaraldehyde tissue adhesive. The myometrial composition may be used for immediate cardiomyoplasty implantation, or alternatively for storage as a source of mymometrial tissue to be used for subsequent implantation. In the latter case, the tissue may be freeze-dryed using known techniques.

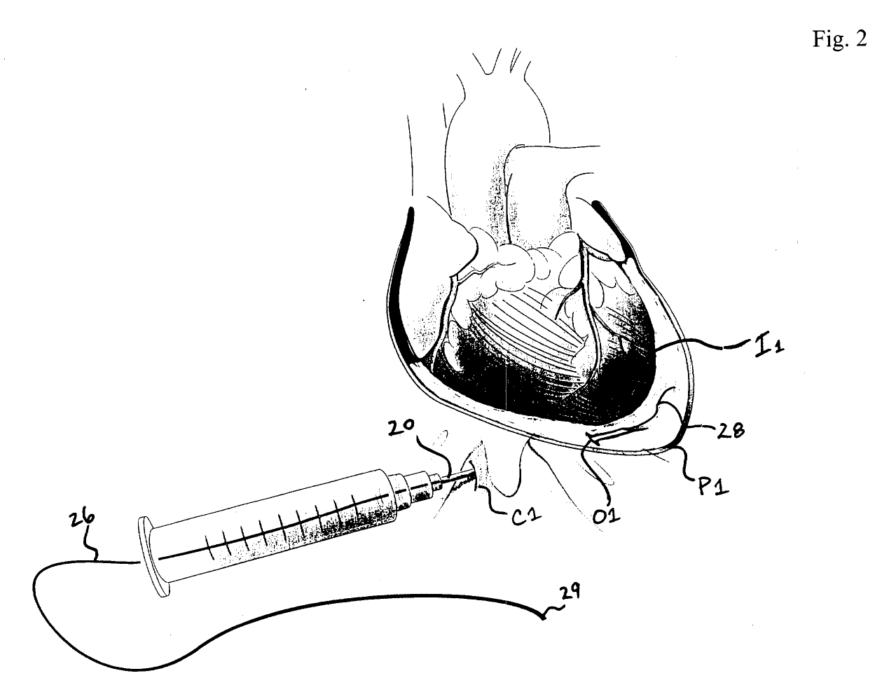

As shown in FIG. 2-4, in accordance with the invention, a myometrial tissue implantation is performed percutaneuosly beginning with insertion of a cannula 20 introduced into a subxyphoidal (or subthoracic) opening C1 in the patient's chest and through an opening O1 in a pericardial sac P1 proximate to an implantation area I1 comprising damaged myocardial tissue. A guide wire 26 is then delivered through the cannula 20 until the proximal end 28 of the guide wire 26 is proximate to the implantation area I1. The distal end 29 of the guide wire 26 remains outside the patient's body.

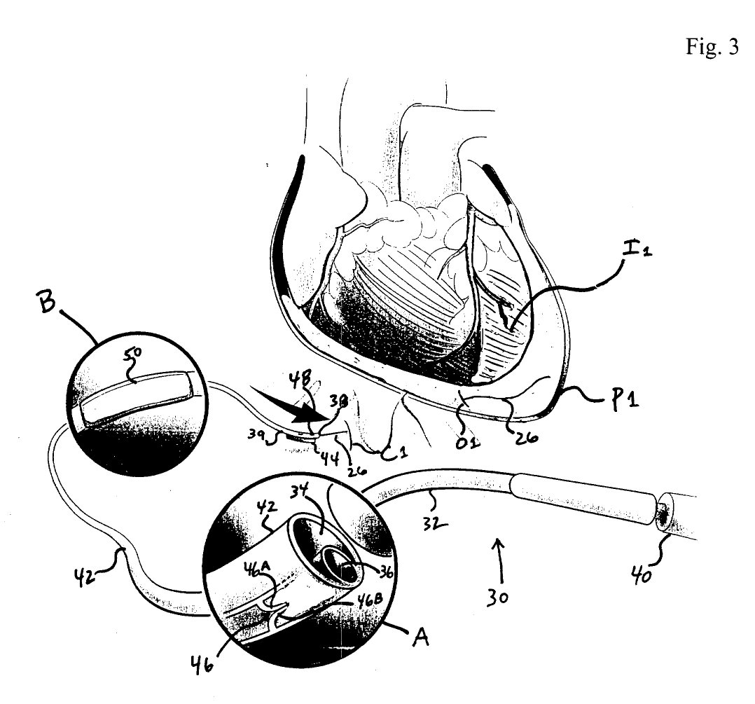

Next, as shown in FIG. 3, a percutaneous tissue implantation device 30 is threaded over the distal end 29 of the guide wire 26 and is slidably positioned proximate to the implantation area I1. The implantation device 30 includes a double lumen tubular body 32, a first tubular interior portion 34 and a second tubular interior portion 36 surrounded by the tubular body 32 (see inset A), a delivery tip 38 at a proximal end 39, a distal end 40, and a central longitudinal axis 42 extending between the delivery tip 38 and the distal end 40. The foregoing components can be made from any suitable biocompatible material that is relatively flexible, such as surgical plastic.

The first interior portion 34 of the implantation device 30 is adapted as a pathway for the myometrial composition. The second interior portion 36 is adapted as a pathway for releasing unsafe pressure within the pericardium during implantation, and comprises a first pressure relief opening 44 on the proximal end 39 of the implantation device 30, a second pressure relief opening (not shown) on the distal end 40 of the implantation device 30, and a pressure relief valve 46 on the central longitudinal axis 42. The first pressure relief opening 44 provides an entrance to the second interior portion 36, and the second pressure relief opening provides an exit. The pressure relief valve 46 can be constructed as a bicuspid valve or the like, made of a suitable biocompatible material such as surgical plastic. The valve 46 comprises a first flap 46A and a second flap 46B that are adapted to separate when pressure within the pericardium rises above a safe level of about 5 mm of mercury during implantation. Thus, material is allowed to pass from the first pressure relief opening 44, into the second interior portion 36 through the open pressure relief valve 46, and out of the patient's body at the second pressure relief opening when an unsafe pressure is reached within the pericardium.

The delivery tip 38 of the implantation device 30 comprises a spray opening 48, adapted for releasing the myometrial compostion from the first interior portion 34. The tubular body 32 preferably includes a stabilizer 50 (see inset B of FIG. 3) adapted to expand and secure the implantation device 30. The stabilizer 50 is constructed as an inflatable bladder made of thin plastic or other highly flexible material, and is expanded using a source such as saline or the like which is supplied through a third interior portion (not shown) on the implantation device 30 or an independent pathway.

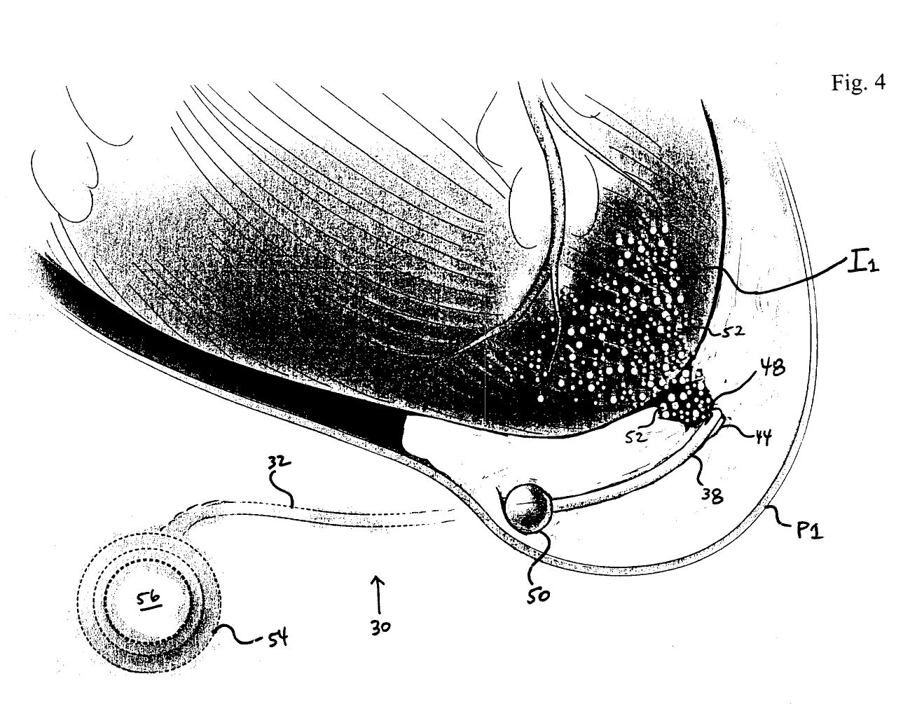

As shown in FIG. 4, with the implantation device 30 properly positioned proximate to the implantation area I1, the stabilizer 50 is expanded within the pericardium PI. Next, a syringe or other solution-carrying device (not shown) is filled with the myometrial composition and is attached to the distal end of the first interior portion 34 of the implantation device 30. The myometrial composition is delivered through the first interior portion 34 and exits as a spray or mist 52 out the spray opening 48 on the delivery tip 38 and onto the implantation area I1.

A port 54 may be positioned under the skin at the subxyphoidal (or subthoracic) opening C1 to allow a regimen of applications of myometrial implantations over a specific period of time. The port 54 is conventional in construction, such as used in chemotherapy. It has a penetrable membrane covering 56 and is adapted to attach to the distal end 40 of the implantation device 30.

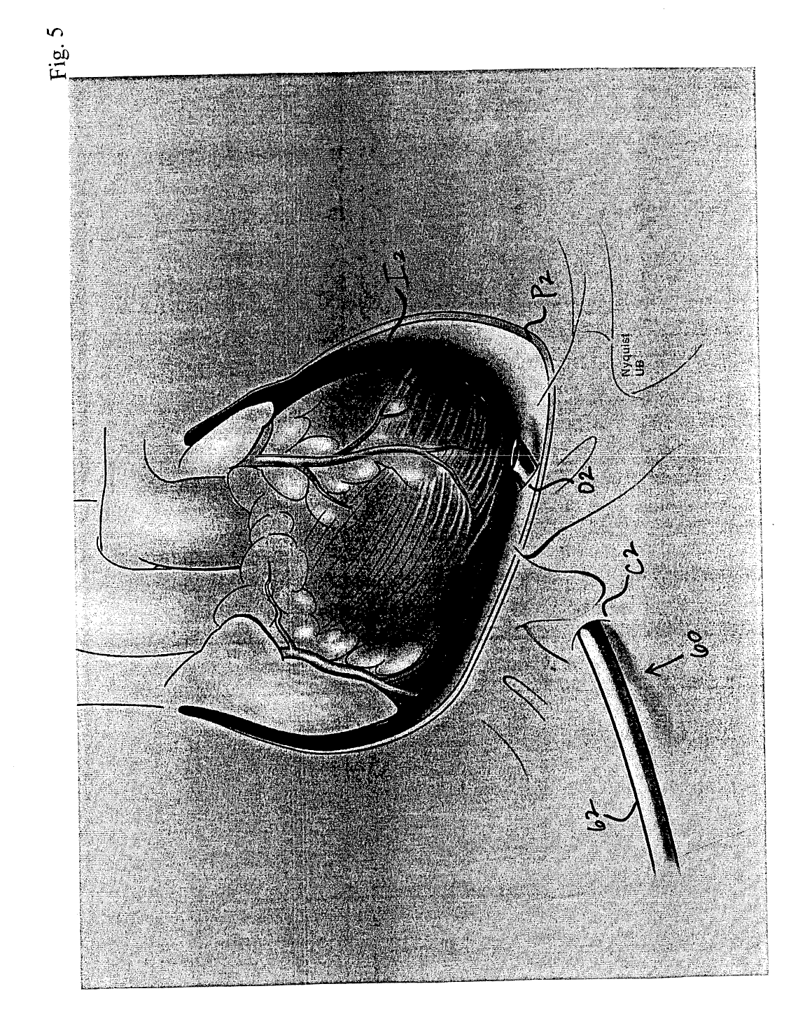

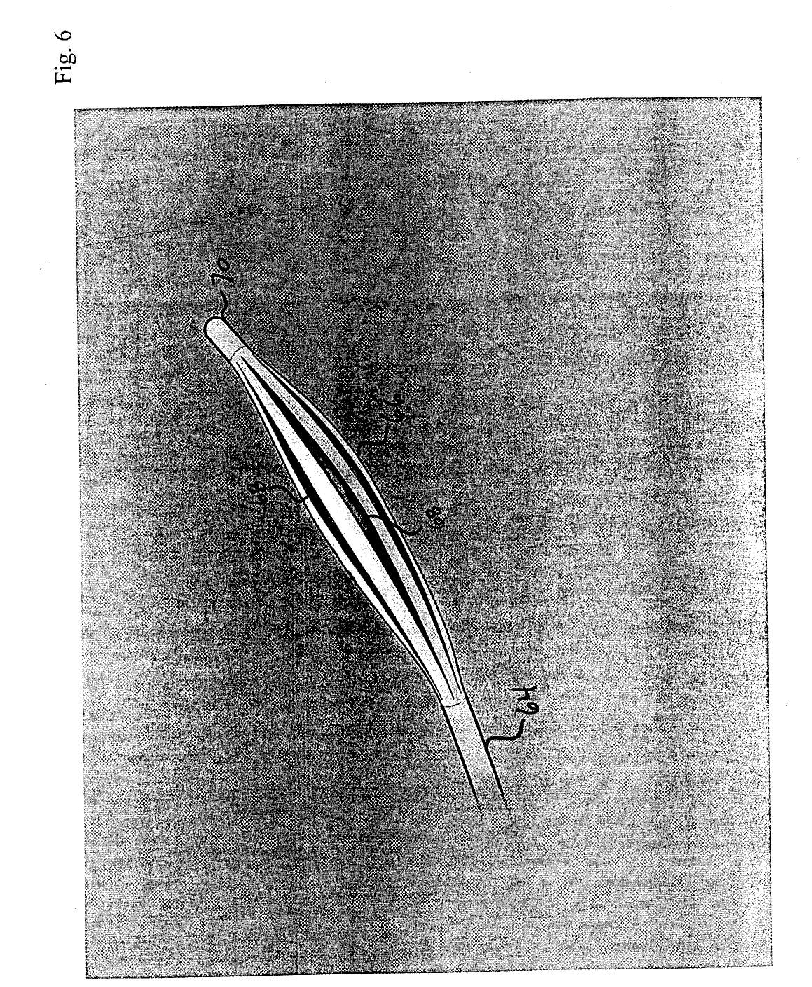

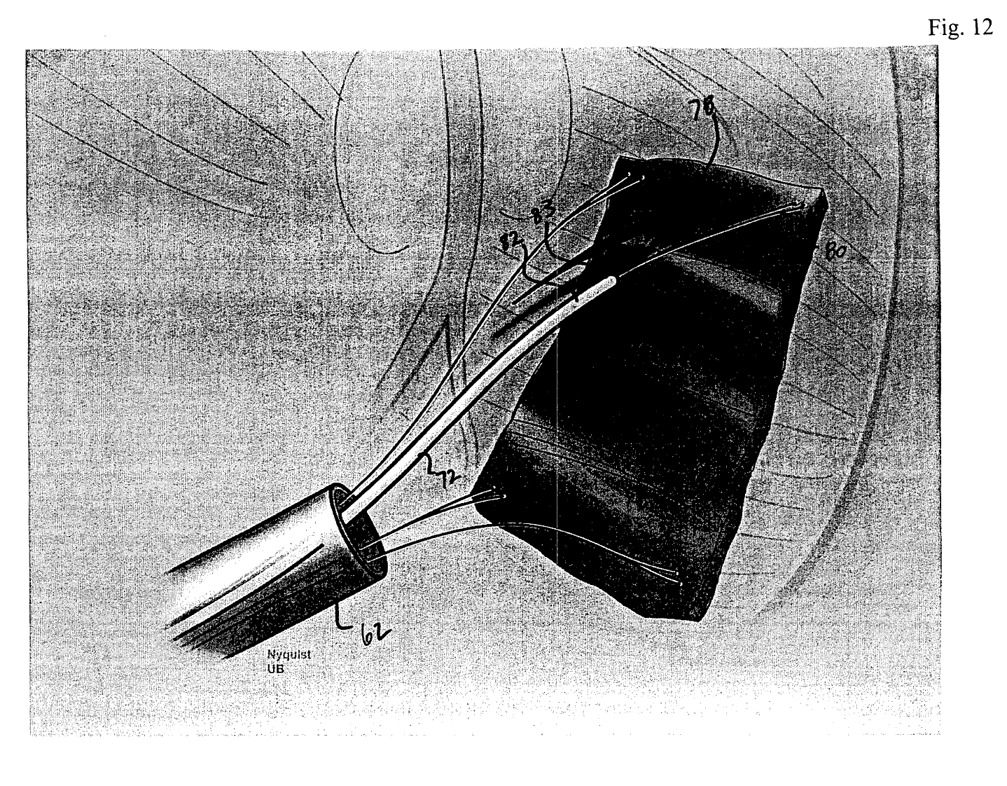

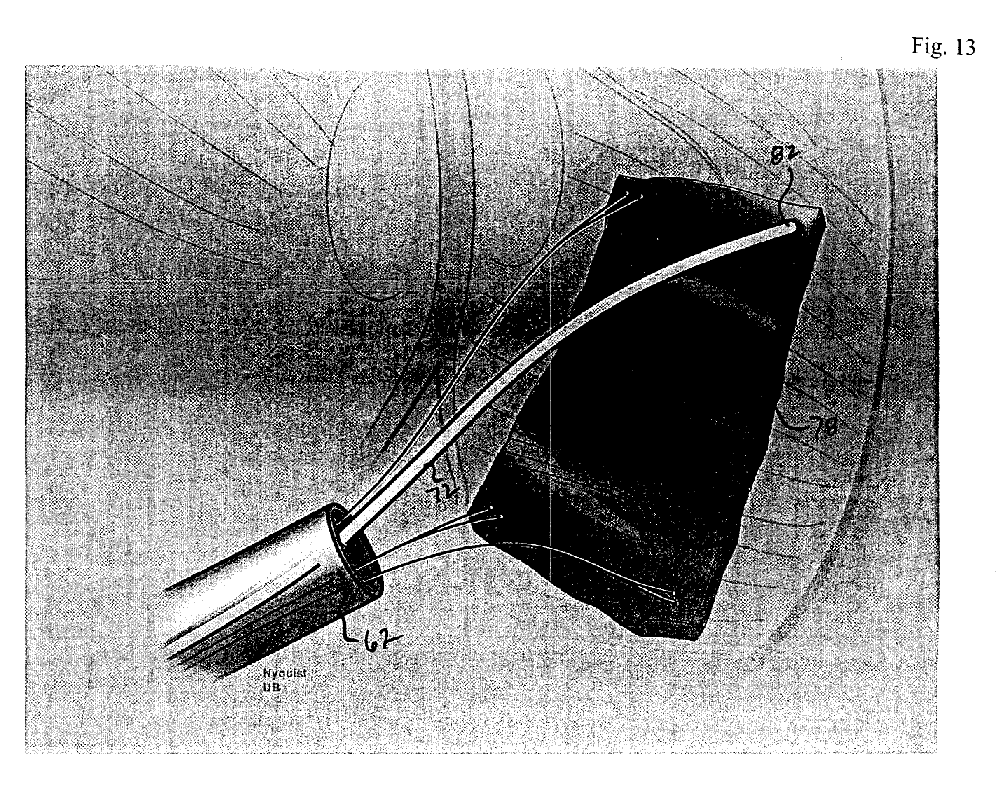

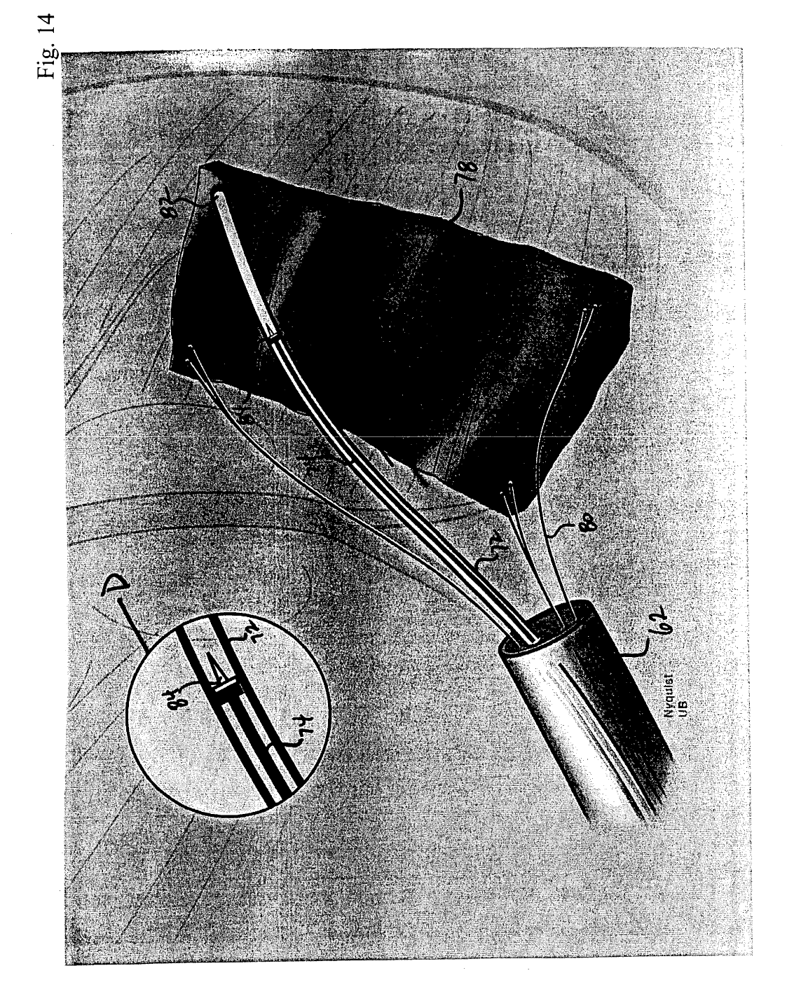

FIGS. 5-17 show a second exemplary myometrial tissue or cell implantation device. The implantation device 60 includes an outer body 62 formed as a hollow tubular lumen, a first tubular lumen inner body 64 having an expandable tip 66 with longitudinal slots 68 on a proximate end 70 (See FIG. 6), a second tubular lumen inner body 72 (See FIG. 12), and a plunger 74 slidably disposed in the second inner body 72 (See inset “D” of FIG. 14). The outer body 62 serves as a pathway for the first inner body 64 and the second inner body 72 respectively. The foregoing components can be made from any suitable biocompatible material that is relatively flexible, such as surgical plastic.

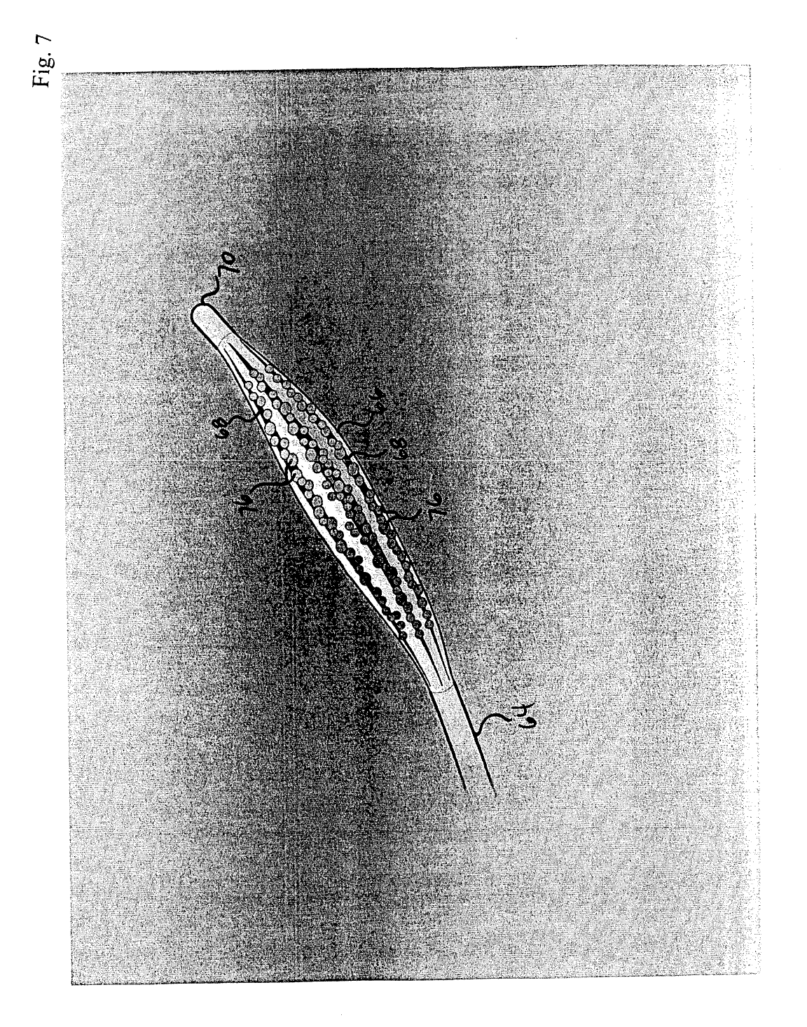

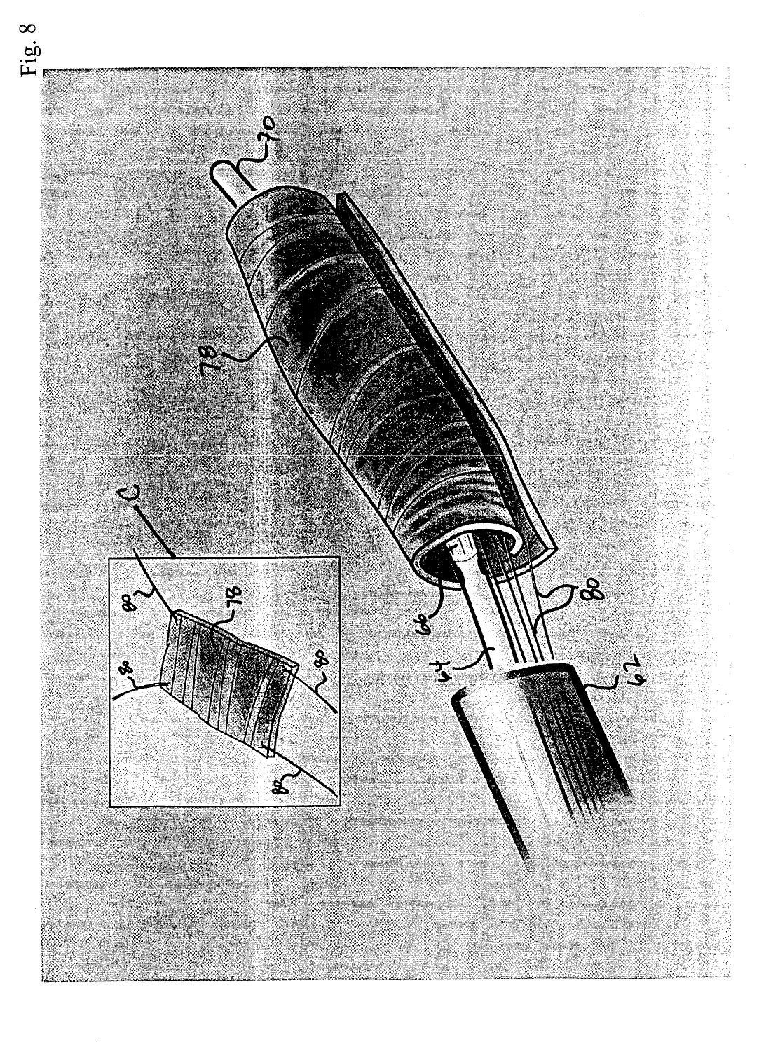

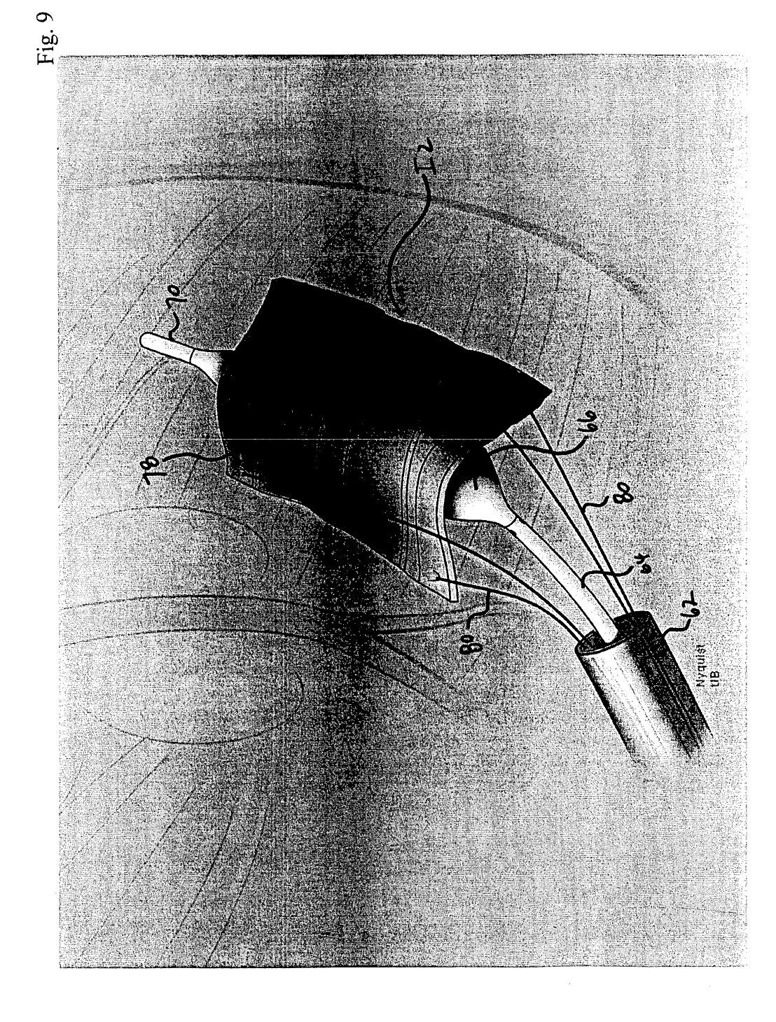

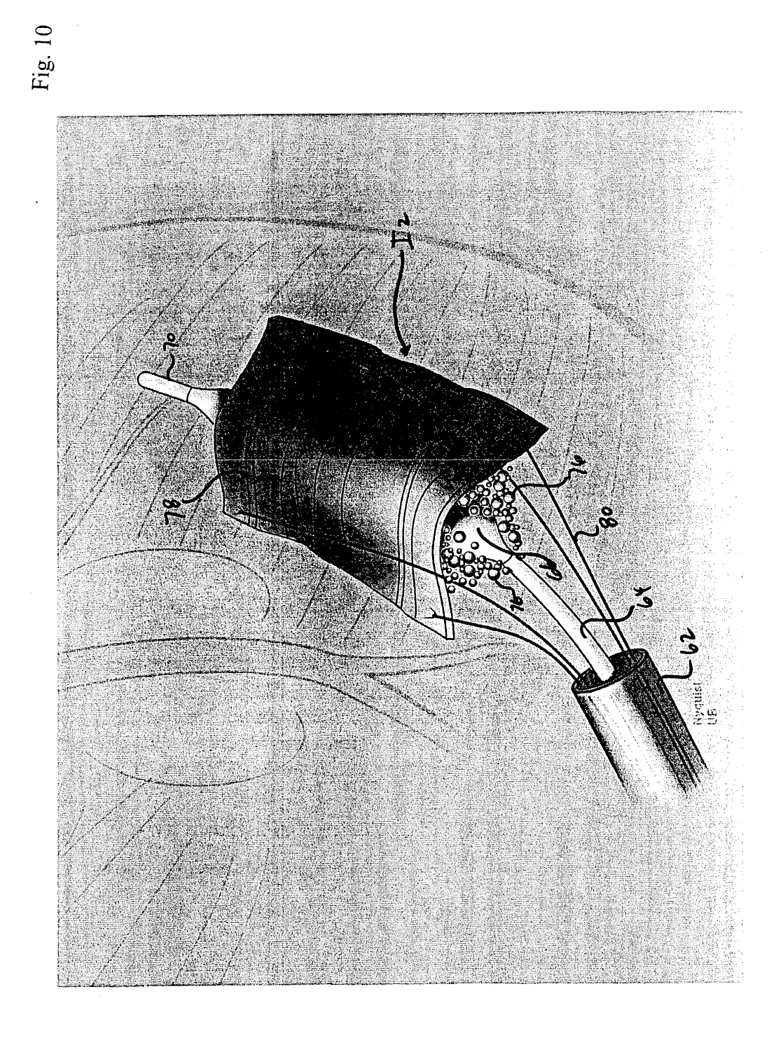

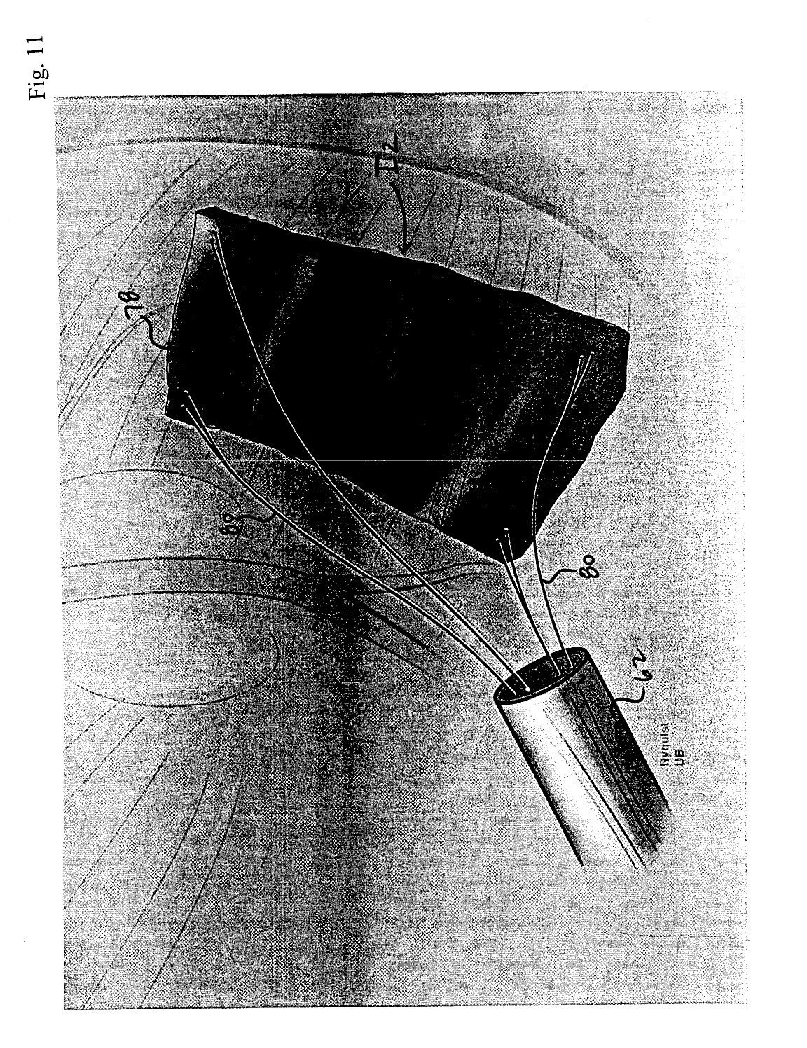

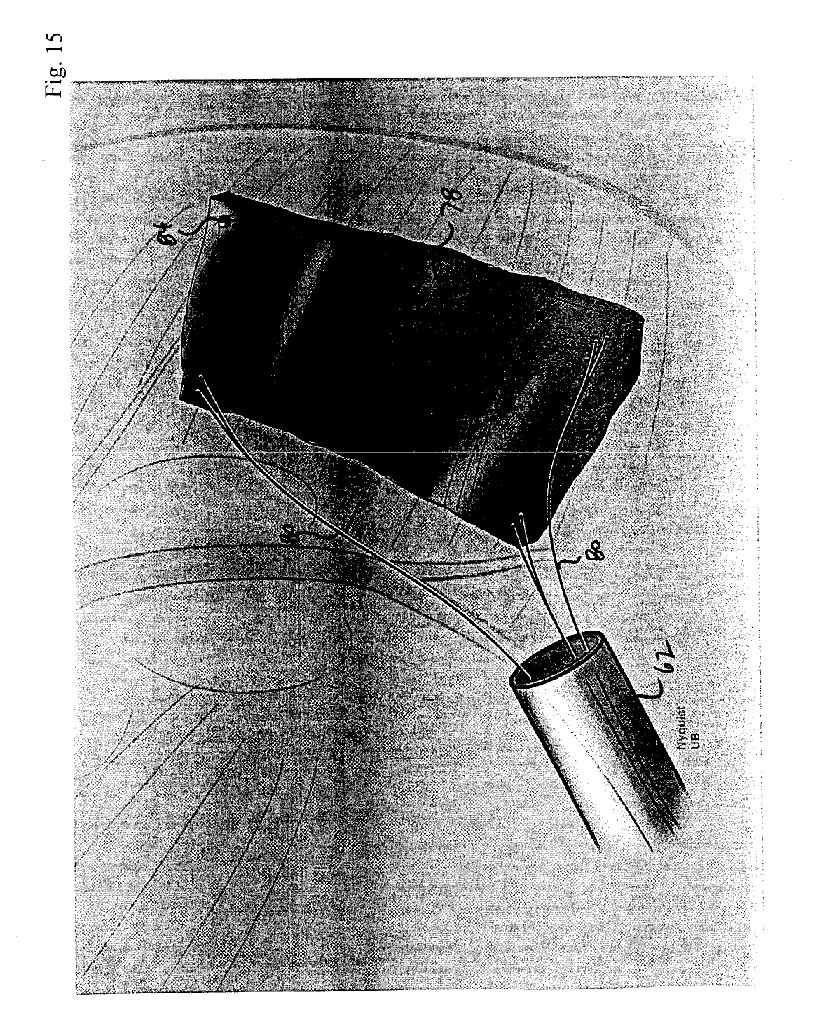

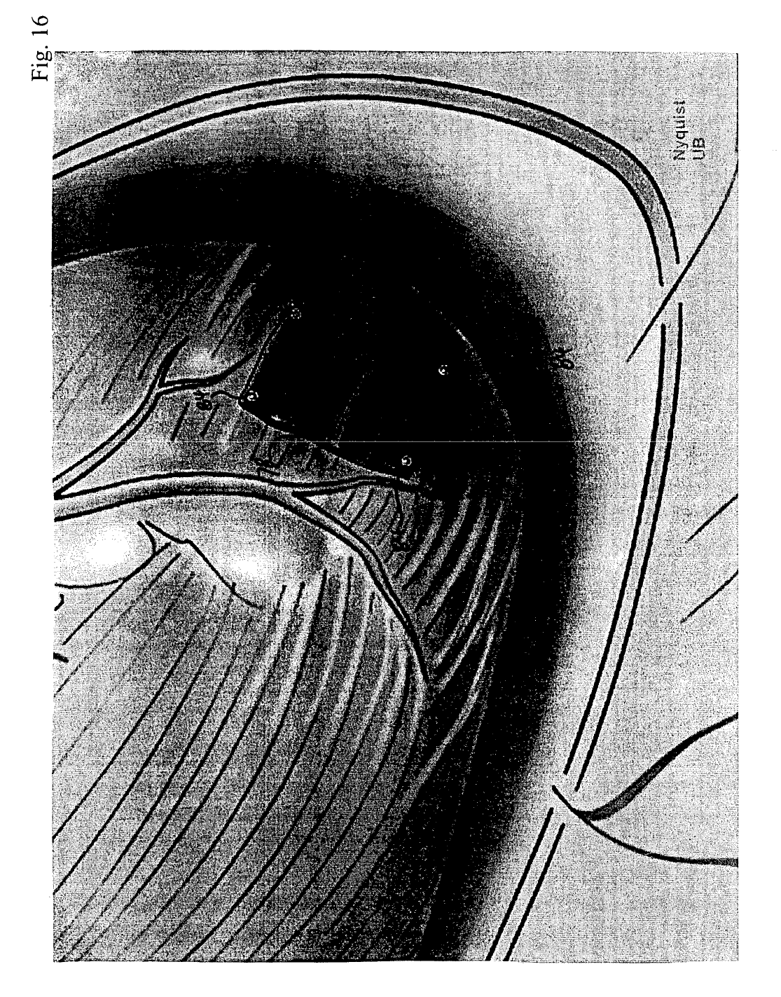

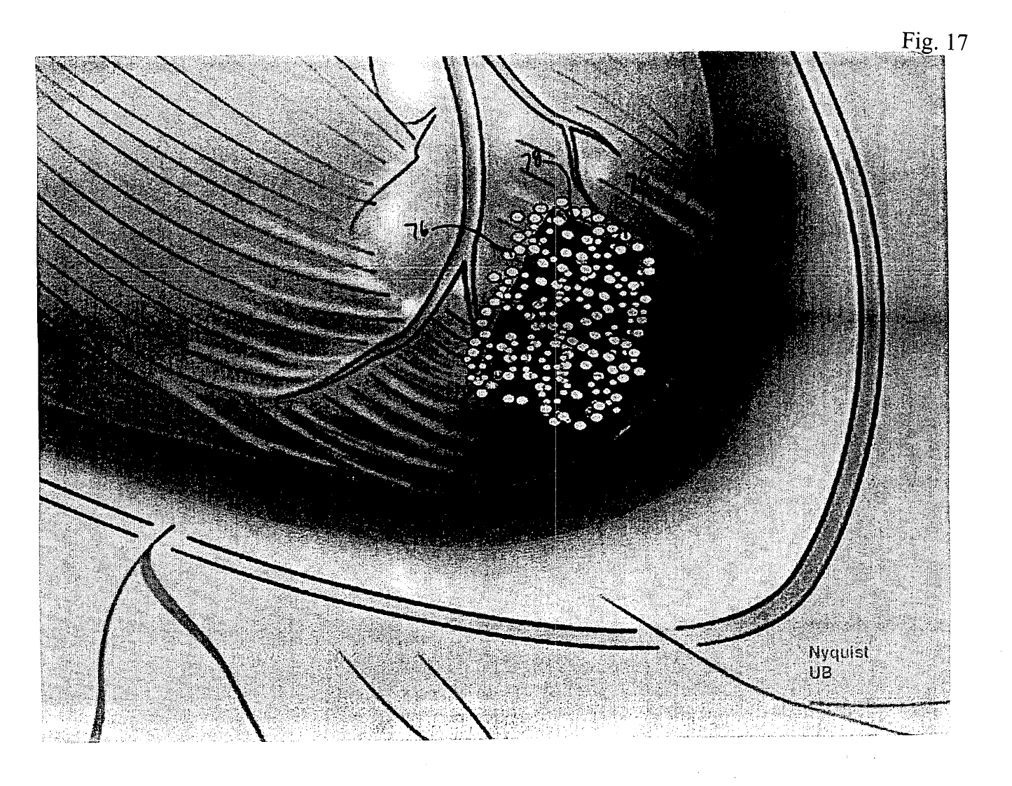

As shown in FIG. 5, a myometrial tissue implantation is performed percutaneuosly beginning by inserting the outer body 62 into a subxiphoidal (or subthoracic) opening C2 in the patient's chest, through an opening O2 in a pericardial sac P2 proximate to an implantation area I2 comprising damaged myocardial tissue. As shown in FIGS. 6 and 7, the first inner body 64 is then made ready by placing a myometrial composition 76 in and around the longitudinal slots 68 and/or by wrapping a thin myometrial tissue patch 78 around the expandable tip 66, as shown in FIG. 8. If a tissue patch 78 is used, it is first releasably sutured (or otherwise secured) with one or more guide wires 80 (see inset C of FIG. 8) to help in positioning the tissue patch 78 onto the implantation area I2 after it is released from the expandable tip 66. During implantation, the first inner body 64 is inserted into and through the outer body 62. When the tip 66 on the first inner body 64 is proximate to the implantation area I2, it is expanded thereby releasing the myometrial composition 76, if it is present, and unwrapping the tissue patch 78, if it is present, as shown in FIGS. 9 and 10. Once the myometrial composition 76 and/or the tissue patch 78 have been dislodged from the first inner body 64, the first inner body 64 is removed from the outer body 62 by pulling on a distal end (not shown) on the first inner body 64. Next, as shown in FIG. 11, in the case where the tissue patch 78 is used, the guide wires 80 are manipulated to position the tissue patch 78 properly onto the implantation area I2. Then, as shown in FIG. 12, the second inner body 72 is threaded over a distal end (not shown) of one of the guide wires 80 and inserted into and through the outer body 62 until it is proximate to the tissue patch 78. A tip 82 at a proximate end 83 of the second inner body 72 is placed against the tissue patch 78, holding it into place on the implantation area I2 as the guide wire 80 is pulled through the tissue patch 78 and out through the second inner body 72. Next, as shown in FIGS. 14 and 15, a securing fastener, such as a tack 84, is pushed through the second inner body 72 by the plunger 74, and is inserted through the tissue patch 78 and into the underlying implantation area I2, securing the tissue patch 78 to the implantation area I2. This process is repeated for each location on the tissue patch 78 having a guide wire 80 attached, as shown in FIG. 16. FIG. 17 shows the tissue patch 78 secured to the implantation area I2 along with the composition 76.

The principles of the present invention have been validated as a result of animal testing. In particular, ten rabbits underwent cardiomyoplasty using myometrial tissue following ligation of the left anterior descending artery. Six of the rabbits had cell patch cardiomyoplasty using myometrial patches and four rabbits had cardiomyoplasty via spray implantation using a myometrial composition comprising myometrial cells. No mortality or morbidity was seen in either group of animals. Histology revealed attachment of transplanted myometrial patches into infarcted myocardium and regeneration of transplanted myometrial cells into scar tissue. Histology also revealed normal nucleus, normal membranes and cytoplasm in the implanted tissue. There was no evidence of degeneration or cell death in the implanted tissue. Echocardiograms revealed contraction of transplanted myometrium muscle synchronous with myocardial contraction.

Accordingly, use of the myometrium as a source for cardiomyoplasty has been disclosed. While various embodiments of the invention have been shown and described, it should be apparent that many variations and alternative embodiments could be implemented in accordance with the teachings herein. It is understood, therefore, that the invention is not to be in any way limited except in accordance with the spirit of the appended claims and their equivalents.

What is claimed is:

1. A cardiomyoplasty method for treating cardiomyopathy comprising:
extracting myometrial tissue from a myometrium;
processing said myometrial tissue for cardiomyoplasty transplantion;
locating an implantation area on a patient's myocardium that contains damaged myocardial tissue; and
applying said myometrial tissue to said implantation area.

2. A cardiomyoplasty method in accordance with claim 1 wherein said myometrial tissue comprises autologous tissue.

3. A cardiomyoplasty method in accordance with claim 1 wherein said myometrial tissue comprises allogeneic tissue.

4. A cardiomyoplasty method in accordance with claim 1 wherein said processing step comprises processing said myometrial tissue into fragments and culturing said fragments.

5. A cardiomyoplasty method in accordance with claim 1 wherein said processing step comprises cutting and/or grinding said myometrial tissue.

6. A cardiomyoplasty method in accordance with claim 1 wherein said processing step includes freeze-drying.

7. A cardiomyoplasty method in accordance with claim 1 wherein said applying step comprises implanting said myometrial tissue into said implantation area without injection.

8. A cardiomyoplasty method in accordance with claim 1 wherein said applying step comprises spraying said myometrial tissue onto said implantation area without injection.

9. A cardiomyoplasty method in accordance with claim 1 wherein said applying step comprises securing said myometrial tissue to said implantation area without injection.

10. A cardiomyoplasty method in accordance with claim 1 wherein said applying step is performed using a noninvasive percutaneous approach.

11. A myometrial composition for cardiomyoplasty comprising:
extracted myometrial tissue; and
a carrying medium.

12. A composition in accordance with claim 11 further including an adhesive medium.

13. A composition in accordance with claim 11 wherein said extracted myometrial tissue is adapted for spraying onto an implantation area on a patient's myocardium that contains damaged myocardial tissue.

14. A composition in accordance with claim 11 further including blood from a patient having said cardiomyoplasty.

15. A composition in accordance with claim 11 further including CDNA vascular endothelial growth factor (VEGF).

16. A composition in accordance with claim 11 wherein said extracted myometrial tissue is in a granulated or ground form.

17. A composition in accordance with claim 11 wherein said extracted myometrial tissue comprises cultured myometrial tissue.

18. A composition in accordance with claim 11 wherein said extracted myometrial tissue is in a freeze-dryed form.

19. A method in accordance with claim 1 wherein said applying step is performed using a device for myometrial tissue implantation in a damaged area of a myocardium comprising:
a tubular double lumen body;
a first pathway on said body;
a second pathway on said body;
a pressure release valve on said second pathway;
an expandable stabilizer portion on said body;
a spray opening on said body proximate to said first pathway adapted to deliver a myometrial cells, granules or solution; and
a port adapted to attach to a distal end of said body.

20. A method in accordance with claim 1 wherein said applying step is performed using a device for myometrial tissue implantation in a damaged area of a myocardium comprising:
a tubular lumen outer body;
a first tubular lumen inner body;
a second tubular lumen inner body;
a delivery tip on said first inner body;
an expandable portion on said delivery tip adapted to carry said myometrial tissue and release said myometrial tissue proximate to said damaged area of a myocardium;
a plunger slidably disposed in said second inner delivery body for delivering a securing member through said second inner body and into said myometrial tissue; and
a port adapted to attach to said outer body.

21. A method in accordance with claim 20 wherein said expandable portion of said implantation device includes longitudinal slots adapted to carry a myometrial composition.
